(19) United States
(12) Patent Application Publication
(10) Pub. No.: US 2004/0132700 A1
(43) Pub. Date: Jul. 8, 2004

(54) Use of fluticasone propionate in the treatment of diseases ameliorated by enhancement of epithelial madrix adhesion such as asthma cystic fibrosis and influenza

(76) Inventors: Joyce Lesley Matthews, Stevenage (GB); Michael Robert West, Stevenage (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/471,687

(22) PCT Filed: Mar. 5, 2002
(86) PCT No.: PCT/GB02/00940

(30) Foreign Application Priority Data
Mar. 12, 2001 (GB) 0106031.8

(51) Int. Cl. A61K 31/573; A61L 9/04; A61K 9/14

(52) U.S. Cl. 514/171; 424/46

(57) ABSTRACT

The invention relates to the use of corticosteroids and fluitcasone propionate in particular for the treatment of diseases which may be ameliorated by enhancement of epithelium/matrix adhesion.

This invention relates to new medical uses for corticosteroids, physiologically acceptable salts and solvates and pharmaceutical compositions thereof, in the treatment of conditions which are ameliorated by enhancement of epitheliall matrix interaction.

The respiratory epithelium forms the first line of defence in the lungs against inhaled particles, noxious gases, allergens, microbial and viral pathogens by acting as both a physical and chemical barrier. The surface epithelial layer contains both ciliated and goblet cells as well as other cells in lower abundance. The integrity of this cell layer is essential for barrier efficacy and is maintained by cell-cell adhesion, mediated primarily through epithelial intercellular junctions. The goblet cells are stimulated by irritants to secrete mucin which is cleared from the airways by the action of the cilia. Tight junctions between the epithelial cells control transepithelial passage of water and solutes, but they also prevent many agents not cleared by mucociliary clearance from penetrating the lung tissue where they could cause serious damage. One of the features of asthma is epithelial damage and sheets of sloughed off epithelial cells are often found in the lumen of the airways. Although the mechanisms behind this epithelial loss are not fully understood, there must be a loss of adhesion between columnar and basal cells and between basal cells and the basement membrane. Repair of areas of sloughed epithelium is relatively fast, involving initially cell migration from the wound, and later cell proliferation so that an epithelial barrier across the wound site is re-established. These cells however are not differentiated (i.e. they have no cilia or mucin) so in the early stages of repair, the normal airway epithelial tissue phenotype is absent. Indeed, these cells display a distinctive ‘wound phenotype’ that is more responsive to, and productive of, inflammatory stimuli. Cigarette smoke, the major cause of COPD, also induces increased permeability of the lung epithelium. It is known that cigarette smoke has a detrimental effect on tight junctions and increases the paracellular permeability of the epithelium. However, the effect of smoke on adhesion of the cells to matrix is not clear. In addition, both bacterial and viral infection of the respiratory tract can cause extensive epithelial damage, this being particularly obvious in the case of influenza. Aside from lung disease, epithelial integrity of the gut is also very important, and in Ulcerative colitis, Chrohns Diseases and NSAID induced ulceration, the epithelial barrier is damaged.

It is probable that successful drug therapy (rather than mere alleviation of symptoms) for diseases like asthma, would need to induce restoration of a normal epithelium and then allow maintenance of that normal epithelium. Steroids seem to be effective in this respect, since the asthmatic epithelial architecture is re-established after a course of corticosteroids. However, it is not currently known what proportion of the therapeutic benefit of steroids in treatment of asthma can be ascribed to its direct effects on the epithelium.

Corticosteroids represent a class of anti-inflammatory compounds which have therapeutic utility topically as creams or ointments or as inhaled preparations. Examples of corticosteroids, which are all encompassed within the present invention, include betamethasone, fluticasone proplonate, budesonide, tipredane, dexamethasone, beclamethasone, fluocinolone, triamcinolone acetonide, mometasone, flunisolide and rofleponide. In particular, the corticosteroid fluticasone propionate (S-fluoromethyl 6α, 9β-difluoro-11β-hydroxy-16α-methyl-17α-propionyloxy-3-oxoandrosta-1,4-diene-17β-carbothiate), described and claimed in UK Patent specification No 2088877B, has proven anti-inflammatory activity and is particularly useful in the treatment of repiratory disorders, particularly asthma.

Apart from inhibiting pro-inflammatory cytokines, little is known about the effect of corticosteroids such as fluticasone propionate on the epithelium. We have discovered that topical administration of a corticosteroid on to epithelial cells has the beneficial effect of protecting the cells against damage, either mechanically or enzyme induced. Treatment of the basolateral surface of bronchial epithelial cells with human sputum elastase, Pseudomonas aeruginosa elastase and porcine pancreatic elastase results in either hole formation in the monolayer due to loss of adhesion of some of the cells to the underlying membrane or to the complete sheet of cells being released for the underlying membrane. Pretreatment of the cells with FP affords some protection against the enzyme induced damage. Equal amounts of mechanical insult result in a smaller wound area in cells which have been treated with FP.

Thus, as a first aspect of the present invention, there is provided the use of a corticosteroid compound in the manufacture of a medicament for the treatment of diseases ameliorated by enhancement of epithelial/matrix interaction.

As an alternative aspect, the invention provides a method of treatment of a mammmalian subject, including human, of diseases ameliorated by enhancement of epithelial/matrix interaction, comprising administration of a corticosteroid compound.

As a preferred aspect of the first or alternative aspect of the present invention, the corticosteroid is preferably fluticasone propionate.

It will be appreciated that reference to treatment is intended to include prophylaxis as well as the alleviation of established symptoms. For example, compounds according to the present invention may be used to reduce exposure of the lung to allergens, irritants or micro-organisms.

Damage to epithelial barrier function has been implicated in the pathology of a number of diseases, including asthma, COPD (chronic obstructive pulmonary disease), cystic fibrosis (CF), viral infections, including influenza, smoke inhalation damage and other infections. Thus, corticosteroids according to the present invention may be used to stabilise the epithelium in asthma or COPD. As an alternative aspect, the corticosteroids of the present invention may be used in the prophylaxis of the symptoms of cystic fibrosis, viral infections and other diseases which cause a trauma to epithelium cells.

As an alternative aspect of the present invention in so far as it relates to the treatment of certain diseases, asthma and COPD are excluded.

While it is possible for the compounds of the invention to be administered alone as the raw chemical, it is preferable to present the active ingredient as a pharmaceutical formulation. Formulations of the corticosteroids for use according to the invention, both for veterinary and for human medical use, comprise the active compound together with one or more pharmaceutically acceptable carriers and optionally any other therapeutic ingredients. The carriers must be pharmaceutically acceptable in the sense of being compatible with the other ingredients of the formulation and not deleterious to the recipient thereof.

The corticosteroids for use according to the invention may be formulated in a conventional manner known in the art for administration by inhalation or insufflation.

For administration by inhalation, the compositions according to the invention are conveniently delivered by conventional means, e.g. in the form of a metered dose inhaler prepared in a conventional manner or in combination with a spacer device such as the Volumatic (Glaxo Group trade mark) device. In the case of a metered dose inhaler, a metering valve is provided to deliver a metered amount of the composition. Spray compositions may for example be formulated as aqueous solutions or suspensions and may be administered by a nebuliser.

Aerosol spray formulations, for example in which the active ingredients are suspended, optionally together with one or more stabilisers, in a propellant, e.g. a halogenated hydrocarbon such as trichlorofluoromethane (propellant 11), dichlorodifluoromethane (propellant 12), 1,2-dichlorotetrafluoroethane (propellant 114), trichlorotrifluoroethane, monochloropentafluoroethane, chloroform or methylene chloride. Also, so-called ozone friendly propellants, such as 1,1,1,2-tetrafluoroethane (propellant 134a), may be employed. Other suitable propellants include, for example, C₁₋₄hydrogen-containing chlorofluorocarbons such as CH₂ClF, CClF₂CHClF, CF₃CHClF, CHF₂CClF₂, CHClFCHF₂, CF₃CH₂Cl and CClF₂CH₃; C₁₋₄hydrogen-containing fluorocarbons such as CHF₂CHF₂, CHF₂CH₃ and CF₃CHFCF₃; and perfluorocarbons such as CF₃CF₃ and CF₃CF₂CF₃. As an alternative aspect of the present invention in so far as it relates to compositions suitable for inhalation, the CFC-free propellant HFA134-a is excluded.

Altematively, for administration by inhalation or insufation, the compositions according to the invention may take the form of a dry powder composition, for example a powder mix of the active ingredients and a suitable carrier such as lactose. The powder compositions may be presented in unit dosage form in, for example, capsules, cartridges or blister packs from which the powder may be administered with the aid of an inhaler such as the Rotahaler inhaler (Glaxo Group trade mark) or in the case of blister packs by means of the Diskhaler inhaler (Glaxo Group trade mark).

Accordingly, the invention also provides the use of a pharmaceutical composition which comprises a corticosteroid and one or more pharmaceutically acceptable carriers or excipients, formulated for administration by inhalation or insufflation, in the manufacture of a medicament for the treatment of diseases ameliorated by enhancement of the epithelial/matrix interaction.

The compositions may contain from 0.1% upwards, e.g. 0.1-99% of the active material, depending on the method of administration. A proposed dose of the compounds of the invention is 0.25 mg/kg to about 125 mg/kg bodyweight per day e.g. 20 mg/kg to 100 mg/kg per day. It will be appreciated that it may be necessary to make routine variations to the dosage, depending on the age and condition of the patient and the precise dosage will be ultimately at the discretion of the attendant physician or veterinarian. The dosage will also depend on the route of administration and the particular compound selected.

The corticosteroids of the present invention may be administered alone. Altematively they may be administered with one or more therapeutic agents and formulated for administration. Appropriate doses will be readily appreciated by those skilled in the art.

For example, the invention provides the use of a corticosteroid or a physiologically acceptable salt thereof, in the manufacture of a medicament for administration either sequentially or simultaneously with a beta-2 agonist for the prophylaxis of a disorder ameliorated by enhancement of epithelial/matrix adhesion. Examples of other suitable beta-2 agonists indude, but are not limited to, salbutamol, formoterol and salmeterol

Combinations of the present invention may also be used to prevent or reduce bacterial infection in the lung. Thus, as a further aspect, there is provided the use of a corticosteroid or a physiologically acceptable salt thereof, in the manufacture of a medicament for administration either sequentially or simultaneously with an anti-bacterial compound for the prophylaxis of lung bacterial infection. Suitable anti-bacterial agents may be selected from a sulphonamide (e.g. sulphamethoxazole), a diaminopyrimidine (e.g. trimethoprim), a combination of a sulphonamide and a diaminopyrimidine (e.g. sulphamethoxazole-trimethoprim), a quinolone, particularly a fluoroquinolone (e.g. ciprofloxacin, levofloxacin and trovafloxacin), a nitroimidazole, a penicillin (e.g. amoxycillin), a cephalosporin (e.g. cefuroxime or ceftazidime), a glycopeptide (e.g. vancomycin), a tetracycline, an aminoglycoside, chloramphenicol, or a macrolide.

The invention will now be described with reference to the following non-limiting examples in which:

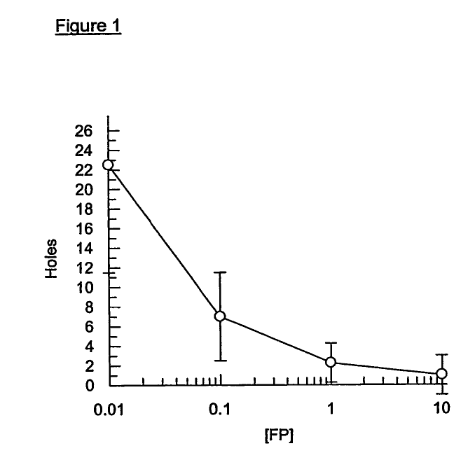

FIG. 1 shows the protective effect of FP against human sputum elastase induced hole formation in monolayers of 16HBe14o- cells;

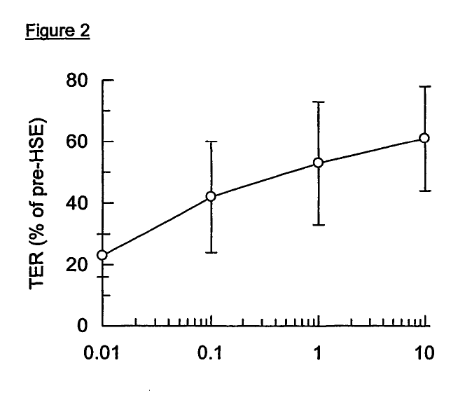

FIG. 2 shows the protective effect of FP on loss of TER of 16HBe14o- cells induced after 24 hour incubation with HSE;

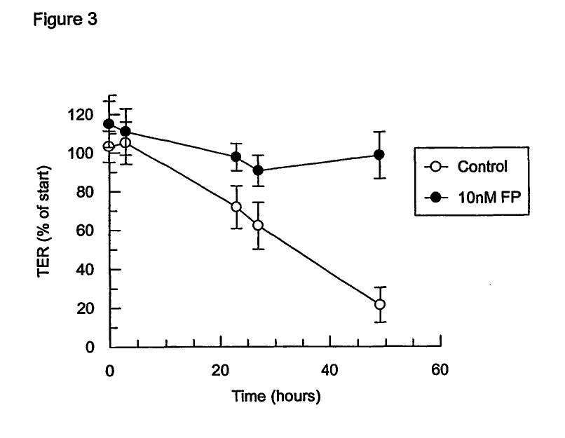

FIG. 3 shows protective effect of FP against human sputum elastase induced loss of TER of primary bronchial epithelial cells;

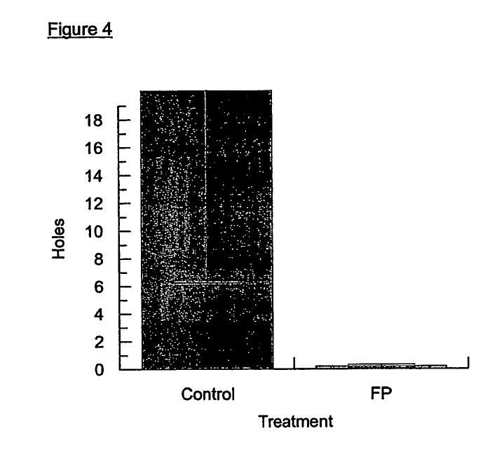

FIG. 4 shows the protective effect of FP against Human sputum elastase induced hole formation in human primary bronchial epithelial cells;

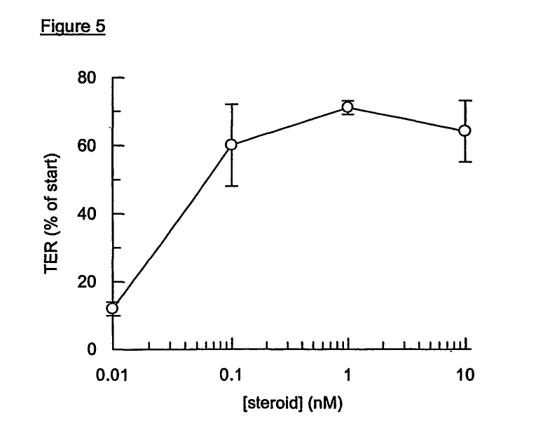

FIG. 5 shows the protective effect of FP against psudomonas elastase induced loss of TER of 16HBe14o- cells;

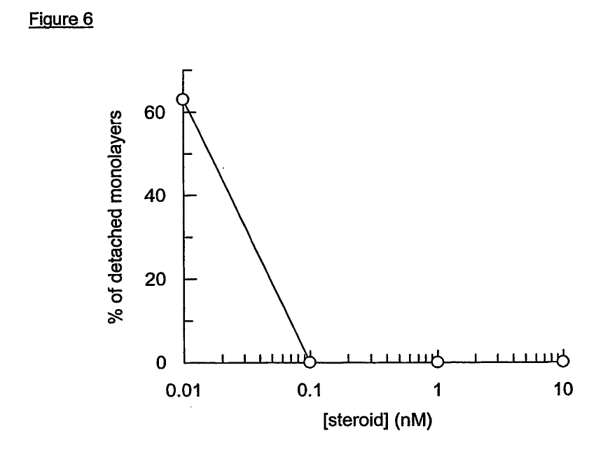

FIG. 6 showing protective effect of FP against pseudonmonas elastase induced detachment of 16 HBe14o- monolayers form the underlying membrane; and

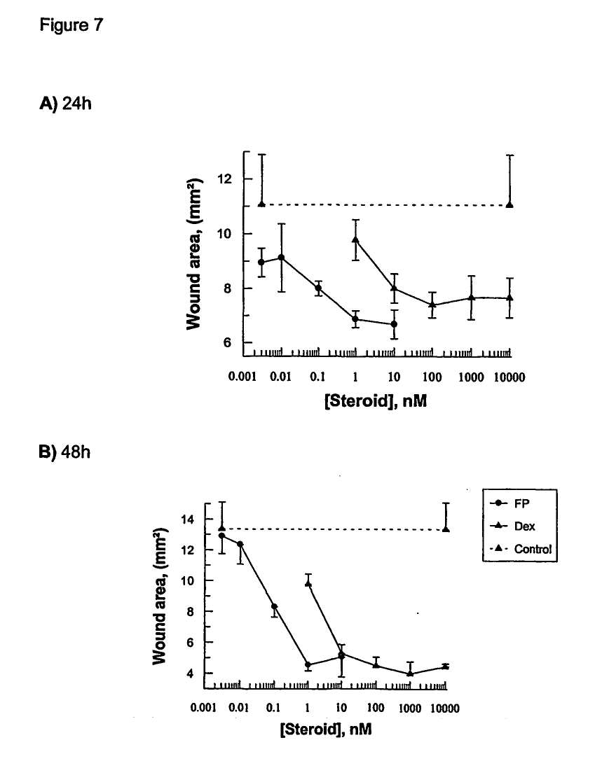

FIG. 7 shows the effect of FP on 16HBE epithelial wound area.

### EXPERIMENTAL DETAILS

16HBe14o- cells were grown on transwell clear membranes until they formed an electrically tight monolayer. In the absence of serum, the cells were treated for 24 hours with FP or vehicle. 5 μg/ml human sputum elastase (HSE) or 5 μg/ml Pseudomonas Aeruginosa elastase (PAE) was then added to the basolateral surface of the cells. The transepithelial resistance (TER) and the morphology of the monolayers were monitored over the next 24 hours.

When HSE was added to the basal surface of the both the 16HBE14o- and primary bronchial epithelial cells it caused a lowering of TER and formation of holes in the cell monolayers. Treatment with FP caused a reduction in the rate of hole formation. When the cells were treated with PAE, the monolayer detached from the underlying membrane as a complete sheet. It took 4 hours for all of the control cultures treated with PAE to become detached whereas it took 8 hours for all of the FP pretreated cultures to become detached.

To study the effect of FP on protection from mechanical damage, multiple concentric wounds were generated in multiwell plates, thus achieving a large total wound length for analysis. 10⁴ cells in 0.2 ml of growth medium containing 5% steroid-free FCS were added to wells in 96 well plates and cultured until confluent with regular changes of growth medium. After confluence, the medium was replace with fresh medium ±various concentrations of steroid, as indicated in FIG. 7. Cultures were incubated for a further 24 h or 48 h before wounding. After wounding, cultures were rinsed in PBS, fixed at room temperature in methanol/acetone (50/50), air dried and then stained with 0.1% crystal violet. Wound areas were determined using a Leica Q550IW image analysis system. Wound sizes are shown in FIG. 7. Control cultures have the largest wound area, but there is a dose-related decrease in wound size for both dexamethasone or FP treated-cultures. The steroid effects were obvious at 24 h, but even more marked at 48 h. At the latter time point, control wound size had increased, whilst the highest dose of either steroid had reduced wound size, compared to wounds after 24 h exposure. The data suggests that steroids are either reducing intercellular adhesion, or enhancing epithelial cell matrix adhesion. In the control cultures, tearing of the epithelium from the dish surface beyond the area of direct contact with the needle occurs producing a wound width that is much greater than the needle width. In contrast, in steroid treated cultures, wounds become progressively smaller with increasing steroid concentration until the damage area is limited to the epithelium that has had direct contact with the needle. Since we know that steroids enhance 16HBE14o-intercellular tight junction function, we believe that this data reflects a steroid-induced increase in epithelial-matrix adhesion.

### SUMMARY

Results show that treatment of bronchial epithelial cells with FP protects against both elastase induced detachment of cells from matrix and also from mechanically induced damage. It is assumed that this protection is a result of upregulation of either expression and/or activation of adhesion molecules attaching the cells to the matrix, or to changes in matrix deposition.

1. Use of a fluticasone propionate, or a physiologically acceptable salt or solvate thereof, in the manufacture of a medicament for the treatment of diseases ameliorated by enhancement of epithelial/matrix adhesion.

2. Use according to claim 1, with the proviso that the diseases do not include asthma or COPD.

3. Use of a pharmaceutical composition suitable for inhalation comprising fluticasone propionate, or a physiologically acceptable salt or solvate thereof and a CFC-free aerosol propellant, in the manufacture of a medicament for the treatment of diseases ameliorated by enhancement of epithelial/matrix adhesion.

4. Use according to claim 3 where the composition does not include HFA134-a propellant.

5. Use of fluticasone propionate or a physiologically acceptable salt thereof, in the manufacture of a medicament for administration either sequentially or simultaneously with a beta-2 agonist for the prophylaxis of a disorder ameliorated by enhancement of epithelial/matrix adhesion.

6. A pharmaceutical composition comprising a combination of fluticasone propionate and a neuraminidase inhibitor and one or more carriers or excipients.

7. A pharmaceutical composition according to claim 6 where the neuraminidase inhibitor is zanamivir.

8. Use of fluticasone propionate or a physiologically acceptable salt thereof, in the manufacture of a medicament for administration either sequentially or simultaneously with a neuraminidase inhibitor for the prophylaxis of a disorder ameliorated by enhancement of epithelial/matrix adhesion.

9. Use of a dry powder pharmaceutical composition suitable for insufflation comprising fluticasone propionate and lactose, in the manufacture of a medicament for the treatment of diseases ameliorated by enhancement of epithelial/matrix adhesion.
